(19) United States
(12) Patent Application Publication
(10) Pub. No.: US 2015/0000706 A1
(43) Pub. Date: Jan. 1, 2015

(54) METHOD FOR CLEANING DEPOSITS FROM EQUIPMENT THAT HAVE ACCUMULATED DURING A METHOD FOR RECOVERING NMP, BY THE INTRODUCTION OF HOT WATER AND AGITATION

(71) Applicants: Aurelie Alemany, Mannheim (DE); Maximilian VICARI, Limburgerhof (DE); Tanja KIRCHNER, Nierstein (DE); Frank STEGLICH, Gruenstadt (DE)

(72) Inventors: Aurelie Alemany, Mannheim (DE); Maximilian VICARI, Limburgerhof (DE); Tanja KIRCHNER, Nierstein (DE); Frank STEGLICH, Gruenstadt (DE)

(73) Assignee: BASF SE, Ludwigshafen (DE)

(21) Appl. No.: 14/487,500

(22) Filed: Sep. 16, 2014

Related U.S. Application Data
(63) Continuation of application No. 11/721,827, filed on Jun. 15, 2007, now abandoned.

(30) Foreign Application Priority Data
Dec. 16, 2004 (DE) 10 2004 060 654.4

(51) Int. Cl. B08B 9/08; C07D 207/267

(52) U.S. Cl. CPC B08B 9/08; C07D 207/267
USPC 134/22.1

(57) ABSTRACT

A process is proposed for cleaning an apparatus to remove deposits from a process for the recovery of purified N-methylpyrrolidone (NMP) by evaporating NMP from a contaminated NMP stream which is obtained in a process for the extractive separation of acetylene from the reaction mixture of a partial oxidation of hydrocarbons after expulsion of the acetylene as a gas, wherein hot water is passed into the apparatus and is stirred.

The invention relates to a process for cleaning an apparatus to remove deposits from a process for the recovery of purified NMP.

Acetylene is produced industrially predominantly by partial oxidation of hydrocarbons with oxygen in a high-temperature reaction. The production of acetylene by partial oxidation of hydrocarbons is a known BASF process (Sachsse-Bartholome) and is described, for example, in Ullmann's Encyclopedia of Industrial Chemistry, Sixth Edition, 2000 Electronic Release, pages 1 to 5.

Acetylene is obtained from the reaction mixture of the partial oxidation predominantly by selective absorption using a solvent, in particular N-methylpyrrolidone, abbreviated below to NMP, methanol, ammonia or dimethylformamide. After the acetylene has been expelled as a gas from the latent solvent, a solvent stream which comprises oligomers or polymers of acetylene as impurities, usually in a proportion of about 0.1% by weight, remains. In the literature reference cited in Ullmann's, it is stated that, in the process for the extractive purification of acetylene with NMP, about 2% of the NMP stream is removed continuously from the process and distilled under reduced pressure in order to minimize the loading of the NMP with polymers of acetylene, the polymers remaining behind as virtually dry residue, which is disposed of, Purified NMP is obtained and is recycled to the extractive purification.

In existing processes, the NMP contaminated with about 0.1% by weight of polymer is first concentrated in a preevaporation under reduced pressure, for example to a polymer content of about 3.5% by weight, and then stirred in an externally heated stirred container at from about 130 to 150° C. until no more NMP vapor can be taken off. Pasty to solid deposits of a residue, which comprises about 65% by weight of solid, in particular polymers of acetylene, and also about 35% by weight of NMP, remain in the stirred container.

According to known processes, the vacuum is broken with nitrogen after the end of the evaporation of NMP and cold water at atmospheric pressure is then introduced into the stirred container. The water extracts NMP from the deposits and is fed to the wastewater treatment. A solid residue remains in the apparatus and has to be manually removed and then incinerated.

Owing to the necessity of the manual removal of the deposits, this process has disadvantages in terms of occupational hygiene. Moreover, costs are incurred for the disposal of the resulting solids.

In alternative processes, the problem of the handling of solids is avoided by concentrating the contaminated NMP only to such an extent that no solid deposits occur. However, such processes have the disadvantage that a smaller proportion of purified NMP can be recovered.

It was accordingly an object of the invention to improve the existing processes for the recovery of NMP which had been used in the extraction of acetylene, in particular to avoid the handling of solids and at the same time to recover a high proportion of purified NMP.

This object is achieved by a process for cleaning an apparatus to remove deposits from a process for the recovery of purified N-methylpyrrolidone (NMP) by evaporating NMP from a contaminated NMP stream which is obtained in a process for the extractive separation of acetylene from the reaction mixture of a partial oxidation of hydrocarbons after expulsion of the acetylene as a gas, wherein hot water is passed into the apparatus and is stirred.

It was surprisingly found that the pasty to solid deposits in the stirred kettle from which NMP was evaporated can be dissolved in a simple manner using hot water. It is particularly advantageous that the process is independent of the consistency of the deposits, which in turn depends to a great extent on the pH, which varies greatly in the system.

It is possible to pass the purified NMP stream from the extractive separation of acetylene directly into the stirred kettle. However, said stream is advantageously first preevaporated under reduced pressure in one or more stages, preferably to a polymer content of about 3.5% by weight.

Hot water is defined predominantly as water at a temperature of from 40 to 180° C., preferably from 50 to 150° C. Water having a temperature of 90° C. is particularly preferred.

Regarding the water quality, there are in principle no restrictions; for cost reasons, low-quality water is preferred.

The residence time of the water in the apparatus with stirring should be at least 5 min and is advantageously in the range from 5 min to 1 h, particularly preferably about half an hour.

In the apparatus, NMP is evaporated until virtually no vapor pressure is measured. Under these conditions, deposits of a pasty to solid residue which still comprises about 35% by weight of NMP, the remainder being polymeric solids, remain in the apparatus. These deposits frequently form in a layer thickness of from 0.1 to 30 cm, in particular from 1 to 10 cm.

The volume of the water passed into the apparatus is advantageously in the range from about 2 to 30 times the volume of the deposits, preferably about 15 times the volume of the deposits.

A solution which passes through a filter paper without leaving a residue and also remains stable on cooling without polymer being precipitated is taken off from the apparatus. The aqueous solution taken off frequently comprises about 2% by weight of material of the deposits and 2% by weight of NMP and has a water-like viscosity in the range from 1 to 5 mPa·s.

The apparatus in which NMP is recovered and which is subsequently freed from impurities is preferably a stirred kettle.

The process therefore has the advantage that contaminated NMP from the extractive separation of acetylene can be substantially concentrated to a solids content of about 65% by weight, and a correspondingly high proportion of recovered pure NMP, without problems occurring thereby with the handling of solids, in particular the manual removal of the deposits, and with the disposal.

The deposits from the apparatus are taken off as aqueous solutions, which also remain stable after mixing with river water for cooling to about 40° C., i.e. exhibit no polymer precipitate, can be passed into wastewater and are readily degraded in a wastewater treatment plant.

The invention is explained in more detail below with reference to a drawing and a working example.

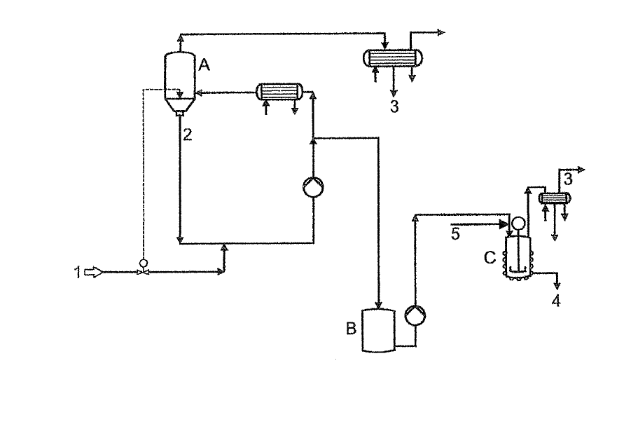

FIG. 1 shows the diagram of a plant for carrying out the process for the regeneration of NMP, reference being made to the modifications of the plant for the process according to the invention compared with the plant from the prior art in the description of the figure.

Stream 1, contaminated NMP from the extractive separation of acetylene, is concentrated in a preevaporator A under reduced pressure, collected in a reservoir B and then fed to a stirred container C having external tracer heating. urified NMP, stream 3, is taken off in vapor form from the stirred container C, and a residue 4 of pasty to solid deposits remains.

Purified NMP, stream 3, is likewise taken off from the preevaporator A.

In the process according to the prior art, the residue 4 from the stirred container C is removed manually as a solid.

In comparison, in the process according to the invention, hot water, stream 5, is passed into the stirred container C, dissolves the residue with stirring and is discharged as aqueous solution.

### COMPARATIVE EXAMPLE

A stream of contaminated NMP from the extractive separation of acetylene from the reaction mixture of a partial oxidation of hydrocarbons, comprising 0.1% by weight of impurities, in particular oligomers and polymers of acetylene, was fed as stream 1 to a plant, as shown schematically in FIG. 1. Stream 1 was concentrated in a preevaporator A under reduced pressure of about 200 mbar to a solids content of about 3.5% by weight, which was taken off as stream 2. Purified NMP, stream 3, was taken off via the top and preferably recycled to the extractive separation of acetylene.

Stream 2 was stored in a reservoir having a capacity of 3 m³ and was fed therefrom batchwise to a stirred kettle C which was heated by an external tracer heating with 4 bar steam to an internal temperature of about 130 to 150° C. A cylindrical stirred kettle having the following geometry was used: kettle diameter 2 m, kettle height 2.6 m and capacity 4 m³. The stirred kettle C was stirred with a horizontal stirrer blade which was mounted at a small distance, about 2 mm, from the bottom of the stirred kettle. From the stirred container C, NMP purified in the form of a gas (likewise stream 3) was taken off from the upper region of the stirred kettle C until a vapor pressure is no longer measured in the stirred kettle C.

The vacuum in the stirred kettle C was broken with nitrogen, and then cold water was passed in for cooling and was discharged after 1 h. Polymer fragments remained in the stirred kettle C and had to be removed manually and incinerated.

The elemental analysis and analysis of the calorific value of the deposits, which may be designated as carbon-like polymer, has led to the following results:

| | |
|---|---|
| Calorific value | 24 870 kJ/kg |
| Sulfur | 1% by weight |
| Hydrogen | 6% by weight and |
| Nitrogen | 4.2% by weight. |

### WORKING EXAMPLE (ACCORDING TO THE INVENTION)

Up to the concentration of the stirred kettle C until a vapor pressure can no longer be measured, the process according to the invention for the recovery of NMP from the contaminated NMP stream from the extractive separation of acetylene does not differ from the prior art process described in the comparative example.

The removal of the deposits from the stirred kettle C after the concentration of the NMP stream to a residual NMP content of about 35% by weight was, however, effected as follows, in contrast to the process from the comparative example: about 2 m³ of hot water at 90° C. were passed into the stirred kettle C onto the 5 cm high deposits in the stirred kettle C and stirred for about half an hour. The deposits dissolved during this procedure, and an aqueous solution comprising about 2% by weight of material of the deposits and about 2% by weight of NMP, having a water-like viscosity in the range from 1 to 5 mPa·s, was taken off from the bottom of the stirred kettle C.

This solution was introduced into the wastewater in need of treatment and was degraded without problems in a wastewater treatment plant.

In the procedure in practice, the stirred container C was free of deposits even after 20 batches corresponding to the above working example.

1.-9. (canceled)

10. A process for cleaning an apparatus to remove deposits from a process for the recovery of purified N-methylpyrrolidone (NMP) which comprises evaporating NMP from a contaminated NMP stream which is obtained in a process for the extractive separation of acetylene from the reaction mixture of a partial oxidation of hydrocarbons after expulsion of the acetylene as a gas, wherein hot water is passed into the apparatus and is stirred in order to remove deposits, wherein stirring is effected for at least 5 minutes, wherein an aqueous solution with a viscosity of from 1 to 5 mPa·s is taken off from the apparatus, wherein all of the deposits are removed without a manual removal step.

11. The process according to claim 10, wherein the contaminated NMP stream from the process for the extractive separation of acetylene is passed into one or more preevaporation stages before being fed into the apparatus.

12. The process according to claim 10, comprising a layer thickness of the deposits of from 0.1 to 30 cm.

13. The process according to claim 10, comprising a residual NMP content in the deposits of about 35% by weight.

14. The process according to claim 10, wherein the volume of the water passed into the apparatus is from about two to 30 times the volume of the deposits.

15. The process according to claim 10, wherein an aqueous solution comprising about 2% by weight of material of the deposits and about 2% by weight of NMP is taken off from the apparatus.

16. The process according to claim 10, wherein the apparatus is a stirred kettle.

17. The process according to claim 10, wherein the hot water is at a temperature of about 90° C.

18. The process according to claim 10, wherein stirring is effected from 5 min to 1 hr.

19. The process according to claim 18, wherein stirring is effected about half an hour.

20. The process according to claim 12, comprising a layer thickness of the deposits from 1 to 10 cm.

21. The process according to claim 14, wherein the volume of the water passed into the apparatus is about 15 times the volume of the deposits.

22. The process according to claim 11, wherein the hot water is at a temperature in the range from 50 to 150° C.

23. The process according to claim 22, wherein the volume of the water passed into the apparatus is from about two to 30 times the volume of the deposits.

24. The process according to claim 23, wherein an aqueous solution comprising about 2% by weight of material of the deposits and about 2% by weight of NMP is taken off from the apparatus, and wherein the aqueous solution has a viscosity of from 1 to 5 mPa·s.

25. The process according to claim 24, wherein the volume of the water passed into the apparatus is about 15 times the volume of the deposits.

26. The process according to claim 10, wherein the hot water is at a temperature of about 90° C., the volume of the water passed into the apparatus is from about two to 30 times the volume of the deposits, stirring is effected about half an hour, and an aqueous solution comprising about 2% by weight of material of the deposits and about 2% by weight of NMP is taken off from the apparatus.
